(19) United States
(12) Patent Application Publication
(10) Pub. No.: US 2025/0255552 A1
(43) Pub. Date: Aug. 14, 2025

(54) Sensor Protector

(71) Applicant: Glen Alton Collett, SANDY, UT (US)

(72) Inventor: Glen Alton Collett, SANDY, UT (US)

(21) Appl. No.: 19/048,954

(22) Filed: Feb. 9, 2025

Related U.S. Application Data
(60) Provisional application No. 63/552,133, filed on Feb. 10, 2024.

(51) Int. Cl. A61B 5/00

(52) U.S. Cl. CPC A61B 5/6833; A61B 2562/18

(57) ABSTRACT

A protector for a skin mounted sensor, the sensor being of the type that has a hard case mounted on patient and having contact with or into the patient's underlying tissue, and the protector being an adjunct device around the sensor, not attached to the sensor, but serving to hold the sensor in place, protect it against accidental glancing blows from a hard object, protect the sensor's attaching adhesive, and provide advanced warning should the attaching material become abraded throughout extended duration.

This application claims the benefit of U.S. Provisional Patent Application No. 63/552,133 filed Feb. 10, 2024.

### BACKGROUND OF THE INVENTION

This invention is a device to protect a sensor attached to the skin of a patient. Technology has developed sensors that can be affixed to a patient's body, and which communicate information to a reading device. For instance, continuously monitoring a patient's blood glucose and reporting that data to an app on a smart phone. Sensors are applied to the patient's skin, and may have a probe which extends into the tissues of the patient's body beneath the skin. The sensors are made of hard plastic material, forming a case around sensitive electronics. The case may be attached to the skin by adhesive double-sided tape, or the like, affixing the sensor in place, perhaps with the probe being suddenly jabbed through the skin and into the underlying tissue. If the probe is subsequently retracted or moved in relation to its penetration point, it can cause the sensor to fail.

Premature sensor failure can happen because of movement of the sensor in relation to the skin. This may be caused by accidently hitting the sensor against a solid object. Another cause of failure is the loosening of the attaching tape or adhesive. A common result of either of these actions is to cause the probe to partially or fully retract, at which point the sensor fails.

It is known in the prior art to cover a sensor with a piece of adhesive tape or film, with the adhesive tape being of various sizes and configurations. While this purports to be protection for the sensor, it still allows the sensor case to be subject to a glancing blow from a solid object, albeit through the tape. Moreover, the adhesive tape sticks to the sensor itself, and if the protective tape is pulled off it then also pulls off the sensor with it.

An object of the present invention is to provide a support for the sensor to keep it in place.

Another object is to provide a gradual ramp around the sensor so that a glancing contact with a solid object is deflected over the sensor and does not lift the sensor case.

Another object is to provide a secondary adhesive, not attached to the sensor, but which functions to keep the sensor tightly in place.

Yet another object is to provide protection and wear resistance for the adhesive or tape of the sensor.

A further object is to provide an early warning of tape loosening caused by abrasion before that tape loosening would affect the movement of the sensor.

The present invention is a device which is independent of the sensor and serves as an adjunct device to support and protect the sensor, but which has no medical function, neither electronic nor chemical.

### DESCRIPTION OF THE DRAWINGS

FIG. 1 is a perspective view of a prior art sensor, showing it attached to the skin of a patient, for example, on the patient's arm.

FIG. 2 is a perspective view similar to FIG. 1, showing the sensor attached to the skin of the patient, and around the sensor, the sensor protector of the present invention.

FIG. 3 is cross-sectional view of the sensor and sensor protector taken along the center of the assembly, as shown in the cross-section designation 3-3 in FIG. 2.

FIG. 4 is and enlarged view of half of the cross-section view similar to FIG. 3, showing the details of the sensor protector.

FIG. 5 is a view similar to FIG. 4 showing the sensor protector on a curved area of the skin of the patient's body.

FIG. 6 is an exploded perspective view of the parts of the sensor protector.

FIG. 7 is an exploded perspective of a second embodiment of the parts of the sensor protector.

FIG. 8 is a cross-sectional view similar to FIG. 3, showing a third embodiment of the sensor protector.

### DETAILED DESCRIPTION OF THE INVENTION

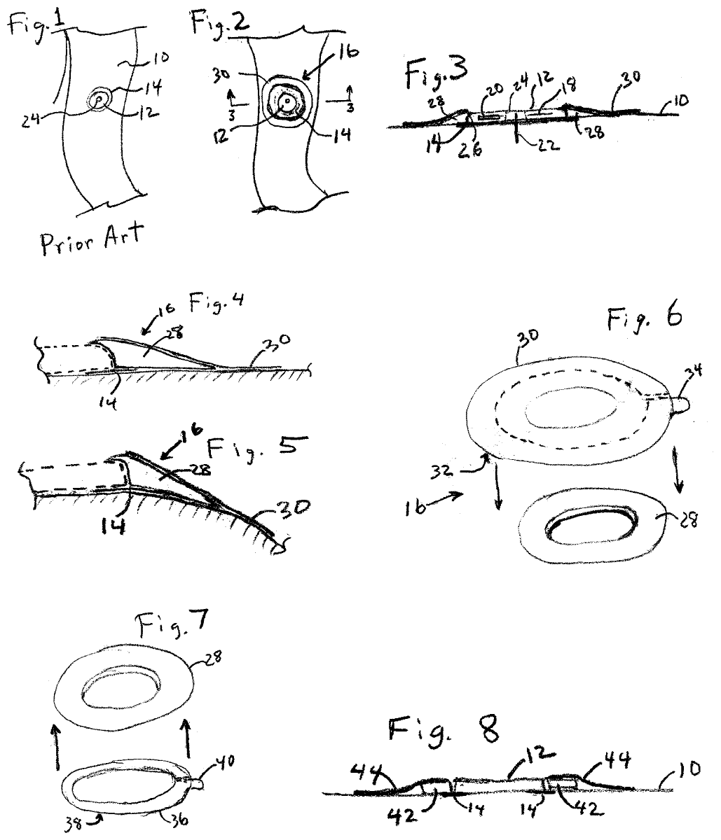

The present invention is a sensor protector for a skin mounted sensor, for example, for a sensor used for monitoring a patient's blood glucose, commonly referred to as a continuous glucose monitor or CGM. FIG. 1 shows the skin of a patient's arm 10. On the skin is mounted a sensor 12, being fastened in place by adhesive, perhaps in the form of double-sided tape 14. It will be understood that the sensor may be of various size and various shape, and may be mounted on the patient's body in any of several locations. The particular size, shape and location shown is for illustrative purposes only. The sensor is normally made of a hard plastic material. As such, it is subject to being hit, snagged, pulled upon, and also subject to abrasion from other contact-skin, clothes or bedding, or wind, sun and water. If a blow to the sensor comes in a glancing manner, the edge of the sensor is subject to being lifted up from the skin. Moreover, the adhesive tape is subject to wear, curling and loss of adhesion due to sweat, water and abrasion.

To combat these effects, the sensor protector of the present invention, as shown in FIG. 2 generally at 16, is placed around the sensor. FIG. 2 also shows a cross-section designation at 3-3. FIG. 3 is the cross-section of the sensor and sensor protector. After the sensor is applied to the skin as shown in FIG. 1, the sensor protector is applied around and over it as shown in FIG. 2, making the assembly shown in FIG. 3.

FIG. 3 shows the sensor 12, which is composed of a hard plastic case, and inside the case are electronics 18, and a battery 20. In the case there is also mounted a probe 22 which is suddenly jabbed beneath the skin 10 when the sensor is applied. Secure and immobile placement of this probe is critical in the continued functioning of the sensor, and dislodgement causes the sensor to prematurely fail. Around the probe may be a small hole 24 through the case allowing atmospheric air to communicate with the skin in the immediate area of the probe. The sensor case is adhered to the skin by an adhesive, perhaps a double-sided adhesive tape 14. This tape extends somewhat beyond the diameter of the sensor case. The top edge of the sensor case may be rounded as shown at 26, in an effort to make the sensor less likely to be snagged. But often this rounding is insufficient to accomplish the intended design function.

The sensor protector 16 of the present invention is shown in detail in FIGS. 4, 5, and 6. The sensor protector is designed to mate with a particular model of sensor, and it will be appreciated that for each model of sensor having a particular size and shape, a corresponding model of sensor protector is used in conjunction. The sensor protector may be described as a flattened torus of material having the cross-section of a wedge rotated about a central axis. It comprises what we will call a donut 28. The donut has an inner surface mating with the sensor case, including conforming to the rounded portion 26 of the sensor case. By this design a small portion of the donut overlaps the sensor case at its outside edge. From the sensor case the donut extends outwardly, with the top of the donut sloping from substantially the height of the sensor case in a wedge shape down to the level of the skin. This forms a ramp which would deflect an object striking towards the side edge of the sensor up and over the sensor.

The donut 28 also covers the sensor adhesive tape 14, which protects it from abrasion. The donut is preferably made of a flexible material, which allows the donut to conform to the surface of the skin, as shown in FIG. 5. This flexibility also allows the skin to change shape, such as would happen if the patient's arm flexes. The sensor case, being rigid, provides for no such potential movement, further stressing the adhesive attaching material. But the donut continues to hold the edges of the sensor case, even if it is flexed in multiple directions.

Covering the donut 28 is an adhesive tape 30 which sticks to the top of the donut and extends outwardly past the narrow edge of the wedge shape of the donut, and which is operable to stick to the patient's skin 10. Thus the sensor protector is fastened in place. The tape 30 does not extend inwardly nor adhere to the sensor case. If the sensor protector comes loose, it could peel off without taking the sensor with it. Meanwhile, the tape 14 holding the sensor onto the skin is protected. By this action, an early warning system is created-if the tape 30 starts to abrade or curl, the sensor protector may loosen, but the sensor itself remains intact and the tape 14 continues to hold the sensor in place.

FIG. 6 shows an exploded view of the sensor protector assembly. Tape 30 is assembled with the flexible donut 28 along the inner margin of tape 30. On the outer margin of tape 30 the underside of the tape has an easy-release film 32 attached to the underside of the tape. This easy-release film has a tab 34 for convenience in removing it when the user is ready to apply the sensor protector. In the manufacturing process the tape 30 is applied to the donut 28 in the direction of the arrows in the figure.

The end user uses the sensor protector in the following manner. First, the sensor 12 is applied to the desired body location according to the sensor manufacturer directions. Probe 22 pierces the skin in the usual manner. The double sided adhesive tape 14 holds the sensor in position. Next the sensor protector 16 is applied over the sensor by centering the donut 28 over the sensor case. Then tab 34 is pulled, removing the easy-release film 32 and exposing the adhesive of tape 30. Tape 30 is pressed onto the skin.

Now the patient goes about normal activities as usual. If an object strikes the sensor protector with a glancing blow, the object rides over the sensor without it taking a significant hit. The sensor protector holds the sensor case firmly against the skin. Tape 14 securely holds the sensor in place and is not subject to abrasion. If the tape 30 becomes loose, the sensor protector 16 can be removed and discarded and another sensor protector can be applied in the same manner, without affecting the security of the sensor. The avoidance of the potential loss of the sensor due to premature failure makes the cost of the additional sensor protector a worthwhile adjunct to the system.

FIG. 7 shows and alternate embodiment of the sensor protector. In this design the donut 28 is the same as previously described. A ring of double-sided adhesive tape 36 is attached to the underside of donut 28 in the direction of the arrows in the figure. The underside of the double-sided tape has an easy-release film 38 attached thereto, with a convenient pull tab 40. The inner diameter of the tape 36 is sized so that it does not stick to tape 14 of the sensor. In applying the sensor protector of this embodiment, the patient places the donut 28 over and around sensor case 12, then pulls the tab 40 to remove the easy-release film, and then presses the donut firmly into place adhering the donut to the skin with the double-sided tape.

A third embodiment of the sensor protector of the present invention is shown in FIG. 8. In this embodiment a donut 42 is sized to have a thickness substantially the same thickness as the sensor, and extends peripherally around sensor 12. Donut 42 is, preferably, made of flexible material. The donut 42 is not necessarily specifically formed in a wedge shape, but it may be made of compressible material so that its outer edge is thinner than its inner edge. An adhesive tape 44 is attached to the top surface of the donut, and extends further outwardly to provide a portion of the adhesive tape which is configured to attach the sensor protector to the skin 10. The tape forms a ramp from the skin to the top of the sensor protector. This may compress the outer edge of the donut, or may press the donut into the skin. The inner surface of the ring of the donut may or may not be designed to exactly conform to the outside edge shape of sensor 12. The use by the patient is very similar as has been described in relation to the previous embodiments.

It will be appreciated that the basic design of a sensor protector according to the present invention applies equally well to all such types of sensors for all purposes, and whether the system is used for human monitoring or for animal monitoring. All shapes and sizes of sensors, locators and drug delivery vehicles are covered by the intent and scope of the present invention. The word sensor is used generically to describe all such devices as may be applied to skin or an animal's hide to have a substantially rigid package attached for an extended period of time. Having described the invention in its preferred embodiments,

I claim:

1. A sensor protector for providing physical protection to a skin mounted sensor, the sensor protector comprising:
a) a donut of material configured to be placed around the sensor, the donut having the cross-sectional shape of a wedge with the inner surface of the donut having a thickness at least as great as the thickness of the sensor, and tapering outwardly to a thinner thickness at the outside periphery of the donut; and
b) an adhesive for holding the donut in position around the sensor.

2. The sensor protector of claim 1 wherein the donut is made of a flexible material.

3. The sensor protector of claim 1 wherein the top outside edge of the sensor is sloped or rounded, and wherein the sensor protector is shaped so that its inside margin conforms to the contour of the sensor, thereby overlapping a portion of the sensor.

4. A sensor protector for providing physical protection to a skin mounted sensor, the sensor protector comprising:
a) a donut of material configured to be placed around the sensor, the donut having a thickness substantially the thickness of the sensor, and
b) an adhesive tape for holding the donut in position around the sensor, the adhesive tape extending beyond the outside of the donut for sticking to the skin, with the top surface of the tape forming a slope from the skin level to an overall thickness at least substantially the thickness of the sensor.
